(19) United States
(12) Patent Application Publication
(10) Pub. No.: US 2011/0150703 A1
(43) Pub. Date: Jun. 23, 2011

(54) TORTUOUS PATH STATIC MIXERS AND FLUID SYSTEMS INCLUDING THE SAME

(76) Inventors: Gustavo H. Castro, Cottage Grove, MN (US); Paul J. Cobian, Woodbury, MN (US)

(21) Appl. No.: 13/054,126

(22) PCT Filed: Jul. 15, 2009
(86) PCT No.: PCT/US09/50704

§ 371 Date: Mar. 8, 2011

Related U.S. Application Data
(60) Provisional application No. 61/081,847, filed on Jul. 18, 2008.

(51) Int. Cl. B01F 5/06; G01N 33/00

(52) U.S. Cl. 422/68.1; 366/336

(57) ABSTRACT

Static mixers and fluid systems incorporating one or more of the static mixers. The static mixers may include one or more channels that provide a tortuous path through a body, wherein fluid flowing through each channel defines a downstream direction through the tortuous path created within the channel. The tortuous path of the channel is preferably formed by a set of flow obstacles protruding into the channel and a set of flow restrictions positioned along the channel between the inlet and the outlet. Each flow restriction has a downstream length over which the open cross-sectional area of the channel decreases and then increases when moving in the downstream direction.

### CROSS REFERENCE TO RELATED APPLICATIONS

This application claims the benefit of U.S. Provisional Patent Application No. 61/081,847, filed Jul. 18, 2008, which is incorporated herein by reference.

Efficient and thorough mixing of materials is a need that is addressed by many different static and dynamic mixers, although many conventional mixers used to mix small volumes of materials often rely on electrical or magnetic fields, long micro-channels or generation of alternating adjacent fluid layers with thicknesses in the micrometer (μm) range (e.g., 25-40 μm) (where the fluid layers are redirected such that they mix). In many instances, however, the mixers suffer from issues such as relatively high pressure drop, limited flow rates, inefficient mixing, etc.

### SUMMARY OF THE INVENTION

The present invention provides static mixers and fluid systems incorporating one or more of the static mixers. The static mixers preferably include one or more channels that provide a tortuous path through a body, wherein fluid flowing through each channel defines a downstream direction through the tortuous path created within the channel. The tortuous path of the channel is preferably formed by a set of flow obstacles protruding into the channel and a set of flow restrictions positioned along the channel between the inlet and the outlet. Each flow restriction has a downstream length over which the open cross-sectional area of the channel decreases and then increases when moving in the downstream direction. The flow obstacles preferably cause fluids flowing through the channel to change direction while the flow restrictions cause the fluid to accelerate and decelerate as it flows past the flow restrictions. The changes in fluid direction and velocity preferably provide a tortuous path that enhances mixing of fluids passing through the channel in the downstream direction.

The open cross-sectional area is the area through which fluid can flow and is determined in a plane that is oriented generally orthogonal to the downstream direction through the channel. The change in the open cross-sectional area can be provided by narrowing the channel in one or more dimensions in that orthogonal plane. For example, the narrowing may occur in the height of the channel (as measured between the bottom surface and the top surface) and/or across the width of the channel between the first and second edges.

It may be preferred that the static mixers of the present invention be capable of mixing small microfluidic volumes of fluids. As used herein, microfluidic static mixers include channels that have, e.g., a cross-sectional area (taken in a plane perpendicular to the downstream flow direction) on the order of 50,000 square micrometers (s-μm) or less. Furthermore, it may be preferred that microfluidic static mixers be capable of mixing fluids with Reynolds numbers in the range of one (1) or less (e.g., Re≦1).

One potential advantage of the mixers of the present invention may include, e.g., a reduction in non-specific binding of analytes to the mixing structures which may be beneficial in connection with biological materials passed through the mixers. In part, the non-specific binding may be reduced by the small surface area to which the biological materials are exposed.

Another potential advantage of the mixers of the present invention is an ability to process smaller sample volumes because of a reduction in the amount of dead volume in the mixers of the present invention.

In some embodiments, static mixers of the present invention may be provided in the form of a multilayer structure that can be manufactured by assembling a base in which the bottom surface of the channel and the set of flow obstacles are formed. The base may also preferably include structure defining the edges of the channel. As a result, the top surface of the channel may preferably be formed by a flat, featureless cover attached to the base over the channel features that are formed in the base. Providing all of the features in base, with the top surface formed by a flat, featureless cover may provide a convenient and economical static mixer structure, particularly where the base may be formed by any suitable process, e.g., molding, etching, sintering, etc.

In one aspect, the present invention provides a static mixer with a mixing structure formed within a body, wherein fluid flowing through the mixing structure defines a flowpath having a downstream direction through the mixing structure from an inlet to an outlet. The mixing structure includes a channel extending from the inlet to the outlet, the channel having a bottom surface and a top surface located opposite the bottom surface, the channel having a first edge and a second edge, wherein the first edge and the second edge extend along a length of the channel on opposite sides of the channel. A set of flow obstacles protrude into the channel, wherein the flow obstacles are positioned at intermediate locations between the first edge and the second edge of the channel. The channel also includes a set of flow restrictions positioned along the channel between the inlet and the outlet, wherein each flow restriction of the set of flow restrictions comprises a downstream length over which an open cross-sectional area of the channel decreases and increases when moving in the downstream direction.

The static mixers of the present invention may include one or more of the following features: the channel may include a set of waveform protrusions positioned along the channel, wherein each waveform protrusion extends across the channel from the first edge to the second edge, and wherein, when moving in the downstream direction, a height of the channel between the bottom surface and the top surface decreases and then increases; the channel may include a set of waveform protrusions positioned along the channel, wherein, when moving in the downstream direction, a width of the channel between the first edge and the second edge decreases and then increases; the open cross-sectional area of the channel may be substantially constant between the waveform protrusions; the bottom surface, the set of flow obstacles, and the set of waveform protrusions may all be formed in a completely integral, one-piece base; the top surface of the channel may be formed in discrete cover that is attached to the base; along the downstream direction, the channel may follow a serpentine flow path between the first side surface and the second side surface; a plurality of flow obstacles of the set of flow obstacles may include a terminal end that does not reach an opposing surface of the channel, such that fluid can pass between the terminal end of the flow obstacle and the opposing surface of the channel, and the terminal surface may include a ramp surface, wherein the ramp surface is inclined relative to the opposing surface, and the ramp surface may have an upstream edge that is lower than a downstream edge; the top surface of the channel may be a flat, featureless surface; the first edge of the channel may include a first side surface located between the bottom surface and the top surface, and the second edge of the channel may include a second side surface located between the bottom surface and the top surface, wherein the second side surface is located opposite the first side surface; the body may be in the form of a flexible body; the downstream direction may follow a curvilinear path that varies in three dimensions; etc.

In a plane oriented orthogonal to the downstream direction of a static mixer of the present invention, the channel may have a maximum height between the top surface and the bottom surface of 250 micrometers or less, 100 micrometers or less, etc.

In a plane oriented orthogonal to the downstream direction of a static mixer of the present invention, the channel may have a maximum width between the first edge and the second edge of 500 micrometers or less; 250 micrometers or less; etc.

In a plane oriented orthogonal to the downstream direction of a static mixer of the present invention, the channel may have a maximum open cross-sectional area of 50,000 square micrometers or less.

In a plane oriented orthogonal to the downstream direction of a static mixer of the present invention, the channel may have a maximum width between the first edge and the second edge and a maximum height between the top surface and the bottom surface, wherein a ratio of the maximum width to the maximum height is 1 or more, 2 or more, etc.

In a plane oriented orthogonal to the downstream direction of a static mixer of the present invention, the channel may have a maximum width between the first edge and the second edge and a maximum height between the top surface and the bottom surface, wherein a ratio of the maximum width to the maximum height is 2 or more and 5 or less.

In another aspect, the present invention may provide an integrated fluid system that includes, in one unitary body, at least the following components: a static mixer according to the present invention; a first chamber located upstream of the static mixer; a second chamber located downstream of the static mixer, and fluid connection channels extending between the static mixer, the first chamber, and/or the second chamber.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. The term “and/or” (if used) means one or all of the identified elements/features or a combination of any two or more of the identified elements/features.

The term “and/or” means one or all of the listed elements/features or a combination of any two or more of the listed elements/features.

The above summary is not intended to describe each embodiment or every implementation of the present invention. Rather, a more complete understanding of the invention will become apparent and appreciated by reference to the following Detailed Description of Exemplary Embodiments and claims in view of the accompanying figures of the drawing.

### BRIEF DESCRIPTIONS OF THE VIEWS OF THE DRAWING

The present invention will be further described with reference to the views of the drawing, wherein:

FIG. 1 is a perspective view of one example of a body containing a static mixer according to the present invention.

FIG. 2 is a plan view of a portion of one exemplary channel that may be used in the mixer of FIG. 1, with the cover removed to expose the interior of the channel.

FIG. 3 is a cross-sectional view of the channel of FIG. 2 taken along line 3-3 in FIG. 2.

FIG. 4 is a perspective view of the channel of FIG. 2.

FIG. 5 is a plan view of a portion of an alternative exemplary channel that may be used in a mixer according to the present invention.

FIG. 6 is a cross-sectional view of the mixer of FIG. 5 taken along line 6-6 in FIG. 5.

FIG. 7 is a plan view of a portion of an alternative exemplary channel that may be used in a mixer according to the present invention.

FIG. 8 is a cross-sectional view of an alternative exemplary channel that may be used in a mixer according to the present invention.

FIG. 9 is a perspective view of a curved body containing a static mixer according to the present invention.

FIG. 10 depicts one exemplary fluid system including two static mixers according to the present invention.

### DETAILED DESCRIPTION OF EXEMPLARY EMBODIMENTS

In the following detailed description of illustrative embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention.

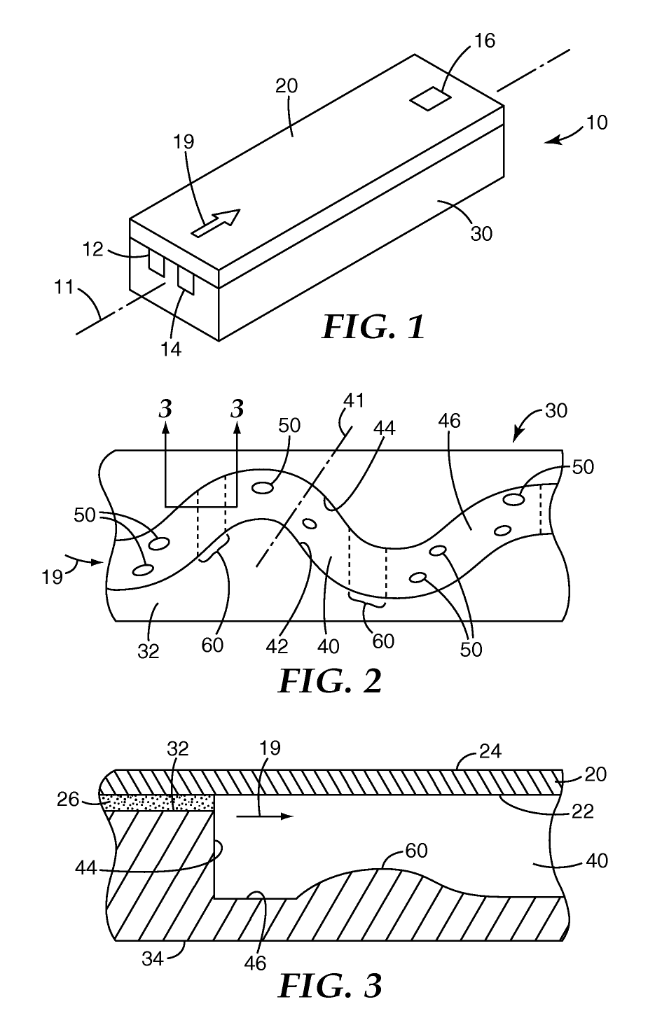

A mixer body 10 containing one exemplary static mixer is depicted in the perspective view of FIG. 1. As depicted in FIG. 1, the body 10 may preferably be in the form of a multilayer structure including two or more layers that provide a cover 20 and a base 30.

The mixer body 10 preferably includes one or more inlets, with the depicted body 10 including a first inlet 12 and a second inlet 14, both of which preferably open into the static mixer located in the body 10. The static mixer in body 10 also preferably includes one or more outlets through which fluids exit the static mixer formed in the mixer body 10, with the body 10 including one outlet 16. Although the static mixer in body 10 includes a pair of inlets 12 & 14 and one outlet 16, the static mixer may (in some embodiments) include only a single inlet and/or one or more outlets.

The inlets 12 & 14 and outlet 16 may define a downstream direction (generally aligned with the longitudinal axis 11) along which fluids passing through the static mixer move. In the view of FIG. 1, the downstream flow direction is represented by arrow 19. In other words, the fluids being mixed in the static mixer in body 10 may enter through the one or more inlets 12 & 14 and, after mixing, exit the static mixer through the outlet 16. In between the inlets 12 & 14 and the outlet 16, the fluids may preferably move in a downstream direction 19 that is generally aligned with the longitudinal axis 11 extending through the body 10.

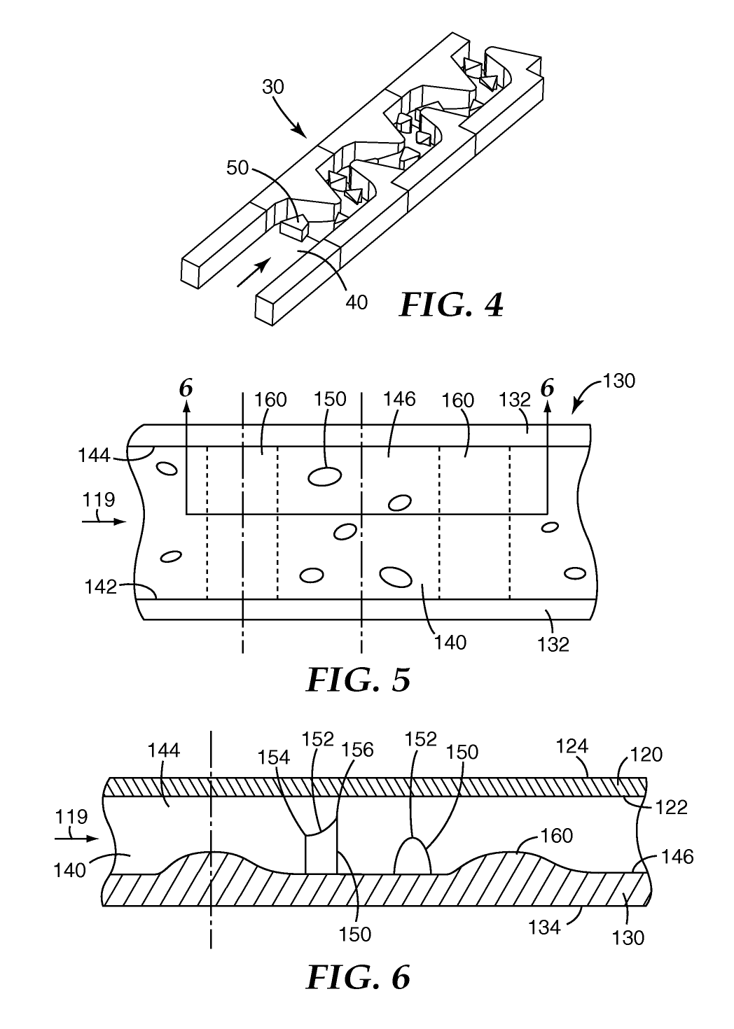

A portion of a channel 40 that may be provided in a mixer according to the present invention is depicted in a plan view in FIG. 2. In some embodiments, it may be preferred that the features of the channel 40 that assist in mixing are all located in the cover 20 or the base 30, with the opposing component enclosing the channel 40 within the body containing the mixer. In the depicted mixer, the channel and its features are formed in the base 30. The cover 20 is removed in the plan view of FIG. 2 and the perspective view of FIG. 4 to expose the features in the channel 40 formed in the base 30. The cover 20 is, however, depicted in connection with the cross-sectional view of FIG. 3.

With reference to FIG. 3, the cover 20 includes an interior surface 22 facing the channel 40 and an exterior surface 24 facing away from the channel 40. The base 30 includes the channel 40 formed therein, including a bottom surface 46 of the channel and the side edge 44 that, in the depicted exemplary embodiment, extends between the bottom surface 46 and the interior surface 22 of the cover 20. The base 30 includes an interior surface 32 that faces the interior surface 22 of the cover 20 and an exterior surface 34 that faces away from the cover 20.

The cover 20 and the base 30 may be attached to each other by any suitable technique or combination of techniques. The exemplary embodiment of FIG. 3 includes adhesive 26 positioned at least between the interior surface 22 of the cover 20 and the interior surface 32 of the base 30.

The channel 40 may take a generally serpentine path as seen in FIGS. 2 and 4, with fluid flow passing through the channel 40 in the downstream direction indicated by arrow 19, although at any particular location in the channel 40 fluid may be moving in any of three dimensions occupied by the channel 40 (i.e., length, width and/or height). The channel 40 includes a first edge 42 and a second edge 44 that extend along the length of the channel 40 on opposites sides of the channel 40. In between the first and second edges 42 and 44, the channel 40 includes a bottom surface 46.

The channel 40 also includes a set of flow obstacles 50 that protrude into the channel 40. In the depicted embodiment, the flow obstacles 50 protrude into the channel 40 from the bottom surface 46. The flow obstacles 50 are preferably positioned at intermediate locations between the first edge 42 and the second edge 44 of the channel 40. The flow obstacles 50 preferably obstruct fluid flow through the channel 40, with each flow obstacle forcing a portion of the fluid to change direction within the channel 40.

The channel 40 may also preferably include a set of flow restrictions 60 positioned along the channel 40 between the inlet and the outlet of the channel 40. Each flow restriction 60 of the set of flow restrictions preferably has a downstream length over which the open cross-sectional area of the channel 40 decreases and increases when moving the downstream direction 19. By restricting the open cross-sectional area of the channel 40, the flow restrictions preferably cause fluid flowing through the channel 40 to change velocity when moving in the downstream direction 19 past a flow restriction 60. Such velocity changes can enhance mixing of fluids passing through the channel 40.

As used herein, the “open cross-sectional area of the channel” is the area, in a plane that is oriented generally orthogonal to the downstream direction through the channel at a selected location, through which fluid can flow through a channel. The edge of one exemplary plane 41 is depicted in FIG. 2 (with the broken lines traversing the channels in FIGS. 5, 6, and 7 also representing exemplary planes similar to plane 41). In a generally rectangular channel, the open cross-sectional area may typically be defined by the height of the channel as measured between the top surface and the bottom surface of the channel and the width of the channel as measured between the opposing side edges. The open cross-sectional area of the channel 40 can be decreased by, e.g., flow restrictions such as flow restrictions 60, that decrease the height of the channel 40 (as, e.g., seen in FIG. 3).

It may be preferred that, as compared to the maximum open cross-sectional area of a given channel, the flow restrictions reduce the open cross-sectional area of the channel by, e.g., about 25% or more, in some instances 50% or more, or even 75% or more.

The flow restrictions 60 depicted in connection with the mixer of FIGS. 2-4 can be described as waveform protrusions that extend across the width of channel 40 from the first side edge 42 to the second side edge 44. The height of the channel 40 between the bottom surface and the top surface progressively decreases to a minimum and then progressively increases when moving in the downstream direction past each flow restriction 60. It may be preferred that the height changes (and, thus, the open cross-sectional area changes) are progressive such that fluid flow passing the flow restriction can be maintained without the risk of forming dead zones, for example, bubble or particle trapping, and closed recirculation. It is not preferred that the flow restrictions result in pooling, collecting or other phenomena that may inhibit mixing of the fluids passing through the channel 40. The use of a waveform protrusion as a flow restriction may provide the preferred progressively decreasing and increasing change in the open cross-sectional areas. It may further be preferred that, except for the flow obstacles, the open cross-sectional area between the flow restrictions remain substantially constant, although this is not required.

It should be understood that although the flow obstacles 50 do reduce the open cross-sectional area of the channel 40, it is preferred that they do not do so as significantly as the flow restrictions. For example, it may be preferred that, within a selected plane oriented generally orthogonal to downstream flow direction, the flow obstacles reduce the open cross-sectional area of the channel (as compared to the maximum open cross-sectional area of the channel) by no more than 10%, in some instances by no more than 25%, or even by no more than 50%.

In another manner of characterizing the differences between flow obstacles and flow restrictions in the channels of mixers of the present invention, the primary function of the flow obstacles may be described as splitting or redirecting the flow, while the primary function of the flow restrictions is to change the velocity of the fluid flowing past the flow restriction.

In the mixer of FIGS. 2-4, it is theorized that, in addition to the flow obstacles 50 and the flow restrictions 60, mixing may be enhanced by the serpentine nature of the channel 40 which may further enhance velocity changes and directional changes in portions of the fluid passing through the channel 40.

It may be preferred that the channel 40 be formed in the base 30, with the cover 20 being provided to form the top surface of the channel 40. In such embodiments, it may be preferred that the bottom surface 46 of the channel 40, the set of flow obstacles 50, and the set of flow restrictions 60 are all formed as a completely integral one-piece base 30. The base 30 and the features provided therein may be formed by any suitable technique, e.g., SMS-based vacuum/thermoformed female tooling, extrusion replication male tool embossing, chemical etching/lithography, two-photon polymerization, laser ablation, etc., and any combination of two or more thereof. Although the features may be provided integrally, they may alternatively be provided as separate components that are assembled to form the desired channel structure.

The top surface of the channel 40 (as formed by the interior surface 22 of the cover 20) may preferably be in the form of a flat, substantially featureless surface. Examples of suitable covers 20 may include films, plates, etc. that are attached to a base 30 by any suitable technique (or combination of techniques) that are capable of sealing the cover to the base such that fluids passing through the channel do not leak into the interface between the cover and the base. Examples of some potentially suitable techniques may include, but are not limited to: thermal bonding, chemical welding, ultrasonic bonding, adhesive bonding (e.g., adhesive layer roll coated mixing structure substrate, adhesive sheet transfer from a backing roll (which may include a post operation for opening any obstructed holes)), etc.

Suitable material or materials used to manufacture the mixer components may include, e.g., polymers (polycarbonates, polypropylenes, polyethylenes, etc.), glasses, metals, ceramics, silicons, etc. The selection of materials may be made based on a variety of factors including, but not limited to, manufacturability, compatibility with the materials to be mixed, thermal properties, optical properties, etc.

Although depicted as a single channel in a single body, the mixers of the present invention may be provided in arrays of two or more channels that are arranged in any suitable configuration, parallel and/or sequentially, as needed to obtain the desired performance in terms of flow throughput, pressure drop, mixing efficiency, etc. For example, two or more separate and discrete channels may be used in parallel to provide two or more paths through a common mixer body, with a single cover enclosing the channels. In another alternative, the channels may be stacked such that, e.g., the exterior surface 34 of one base 30 (see, e.g., FIG. 3) serves as the cover for a lower base, while the upper base includes its own channel. In some mixers channels may be provided in both common bases and in a stacked arrangement, such that a single mixer body may include an array of channels arranged in both X and Z dimensions, where the channels define flowpaths that extend in the Y dimension.

The dimensions of the mixers of the present invention may be selected to obtain the desired flow rates and volumes suitable for the materials to be mixed. In one exemplary embodiment manufactured of a cover 20 and a base 30 (as depicted in, e.g., FIGS. 1-4), the mixer body 10 may have dimensions of about 100 millimeters (mm) in length (measured in the flow direction), 10 mm in width, and 5 mm in height.

The mixers of the present invention may also, or alternatively, be characterized in terms of channel length as measured by the shortest line that travels along the fluid flowpath from the input to the outlet. For example, the channel may be described as having a channel length of 100 mm or less, 50 mm or less, or even 10 mm or less, etc.

Other exemplary dimensions that may be used to characterize the mixers of the present invention may include variations in the height, width, or open cross-sectional area of the channels. For example, as measured in a plane oriented orthogonal to the downstream direction of flow through the channel, the channel may have a maximum height between the top surface and the bottom surface of, e.g., 250 micrometers or less, 100 micrometers or less, etc. In another example, the channel, as measured in a plane oriented orthogonal to the downstream direction of flow through the channel, may have a maximum width between the first edge and the second edge of, e.g., 500 micrometers or less, 250 micrometers or less, etc.

In still another example, the open cross-sectional area of the channel, as measured in a plane oriented orthogonal to the downstream direction of flow through the channel, may have a maximum open cross-sectional area of, e.g., 50,000 square micrometers or less, 12,500 square micrometers or less, etc.

In yet another example, the channel in a mixer of the present invention may have, as measured in a plane oriented orthogonal to the downstream direction of flow through the channel, may have a maximum width between the first edge and the second edge and a maximum height between the top surface and the bottom surface, wherein the ratio of the maximum width to the maximum height may be 1 or more, 2 or more, etc. In some embodiments, the ratio of the maximum width to the maximum height of the channel may be 1 or more and 5 or less.

In certain embodiments, the mixers of the present invention may include variations in the height, width, or open cross-sectional area of the channels that are macro in size. For example, the open cross-sectional area of the channel, as measured in a plane oriented orthogonal to the downstream direction of flow through the channel, may have a maximum open cross-sectional area of, e.g., 5 square millimeters or less, 1.25 square millimeters or less, etc.

A portion of another exemplary mixer according to the present invention is depicted in FIGS. 5 and 6 (where FIG. 6 is a cross-sectional view taken along line 6-6 in FIG. 5). The mixer includes a channel 140 formed in a base 130. Unlike channel 40 described above, the channel 140 does not follow a serpentine path. As with the mixer described in connection with FIGS. 1-4, it may be preferred that the features of the channel 140 that assist in mixing are all located in the base 130, with the cover 120 (see FIG. 6) being provided to serve as a top surface enclosing the channel 140 within the body containing the mixer. The cover 120 is, however, removed in the plan view of FIG. 5 to expose the features in the channel 140.

With reference to FIG. 6, the cover 120 includes an interior surface 122 facing the channel 140 and an exterior surface 124 facing away from the channel 140. The base 130 includes the channel 140 formed therein, including a bottom surface 146 of the channel and the side edge 144 that, in the depicted exemplary embodiment, extends between the bottom surface 146 and the interior surface 122 of the cover 120. The base 130 includes an interior surface 132 that faces (and is attached to) the interior surface 122 of the cover 120 and an exterior surface 134 that faces away from the cover 120. The cover 120 and the base 130 may be attached to each other by any suitable technique or combination of techniques.

The channel 140 may follow a generally straight path with fluid flow passing through the channel 140 in the downstream direction indicated by arrow 119, although at any particular location in the channel 140, fluid may be moving in any of the three dimensions occupied by the channel (i.e., length, width and/or height). The channel 140 includes a first edge 142 and a second edge 144 that extend along the length of the channel 140 on opposites sides of the channel 140. In between the first and second edges 142 and 144, the channel 140 includes a bottom surface 146.

The channel 140 also includes a set of flow obstacles 150 that protrude into the channel 140. In the depicted embodiment, the flow obstacles 150 protrude into the channel 140 from the bottom surface 146. The flow obstacles 150 are preferably positioned at intermediate locations between the first edge 142 and the second edge 144 of the channel 140. The flow obstacles preferably obstruct fluid flow through the channel 140, with each flow obstacle 150 forcing a portion of the fluid to change direction within the channel 140.

It may be preferred that the flow obstacles used in mixers of the present invention do not extend completely from the bottom surface to the top surface of the channel such that at least a portion of the fluid flowing through the channel can pass between a terminal end of the flow obstacles and the opposing top or bottom surface of the channel.

For example, the flow obstacles 150 depicted in FIG. 6 each include a terminal end 152. The left-most flow obstacle 150 seen in the view of FIG. 6 includes a terminal end 152 in the form of a ramp surface that is inclined relative to the top surface of the channel 140 (as formed by the interior surface 122 of the cover 120). The ramp surface includes a leading edge 154 that is lower than the trailing edge 156 (where lower means that the leading edge is further from the opposing surface 122). The right-most flow obstacle 150 seen in FIG. 6 has a different shape and includes a curved or domed terminal end 152. The flow obstacles 150 provided in any one channel in a mixer of the present invention may all have the same general shape or they may take different shapes (as depicted in, e.g., FIG. 6).

The channel 140 may also preferably include a set of flow restrictions 160 positioned along the channel 140 between the inlet and the outlet of the channel 140. Each flow restriction 160 of the set of flow restrictions preferably has a downstream length over which the open cross-sectional area of the channel 140 sequentially decreases and then increases when moving the downstream direction 119. By restricting the open cross-sectional area of the channel 140, the flow restrictions 160 preferably cause fluid flowing through the channel 140 to change velocity when moving in the downstream direction 119 past each flow restriction 160. Such velocity changes can enhance mixing of fluids passing through the channel 140.

The flow restrictions 160 depicted in connection with the exemplary mixer of FIGS. 5 and 6 can be described as waveform protrusions that extend across the channel 140 from the first side edge 142 to the second side edge 144. The height of the channel 140 between the bottom surface and the top surface progressively decreases to a minimum and then progressively increases when moving in the downstream direction past each flow restriction 160. It may be preferred that the height changes (and, thus, the open cross-sectional area changes) are progressive such that fluid flow passing the flow restriction can be maintained without the risk of forming dead zones, for example, bubble or particle trapping, and closed recirculation. It is not preferred that the flow restrictions result in pooling, collecting or other phenomena that may inhibit mixing of the fluids passing through the channel 140. The use of a waveform protrusion as a flow restriction may provide the preferred progressively decreasing and increasing change in open cross-sectional area. It may further be preferred that, except for the flow obstacles, the open cross-sectional area of the channel between the flow restrictions 160 be substantially constant, although this is not required.

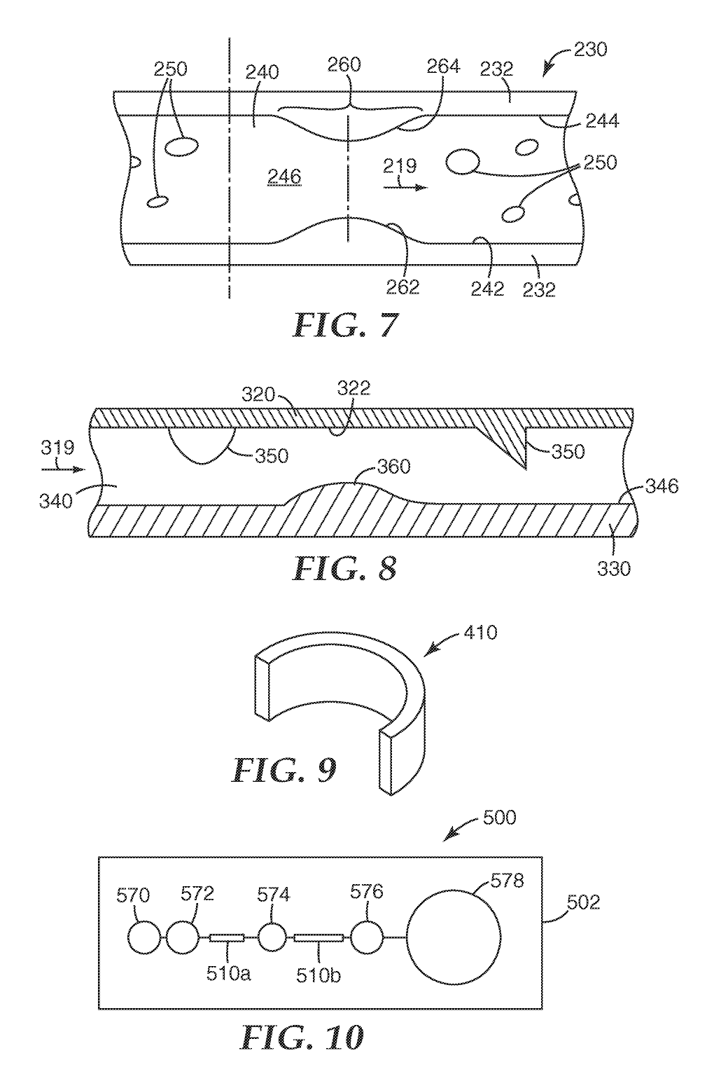

A portion of another exemplary mixer according to the present invention is depicted in the plan view of FIG. 7 (with the cover removed to expose the features in the channel 240). The mixer includes a channel 240 formed in a base 230. As with the mixers described in connection with FIGS. 1-6, it may be preferred that the features of the channel 240 that assist in mixing are all located in the base 230, with the cover (not shown) being provided to serve as a top surface enclosing the channel 240 within the body containing the mixer.

The channel 240 has a bottom surface 246 extending between a first side edge 242 and a second side edge 244. The base 230 also includes an interior surface 232 to which a cover (not shown) could be attached.

The channel 240 may follow a generally straight path, with fluid flow passing through the channel 240 in the downstream direction indicated by arrow 219, although at any particular location in the channel 240 fluid may be moving in any of the three dimensions occupied by the channel (i.e., length, width and/or height).

The channel 240 also includes a set of flow obstacles 250 that protrude into the channel 240. In the depicted embodiment, the flow obstacles 250 protrude into the channel 240 from the bottom surface 246. The flow obstacles 250 are preferably positioned at intermediate locations between the first edge 242 and the second edge 244 of the channel 240. The flow obstacles preferably obstruct fluid flow through the channel 240, with each flow obstacle forcing a portion of the fluid to change direction within the channel 240.

The channel 240 may also preferably include one or more flow restrictions 260 positioned along the channel 240 between the inlet and the outlet of the channel 240. Each flow restriction 260 of the set of flow restrictions preferably has a downstream length over which the open cross-sectional area of the channel 240 decreases and increases when moving the downstream direction 219.

The flow restrictions 260 depicted in connection with the exemplary mixer of FIG. 7 can be described as waveform protrusions 262 and 264 that extend across the width of the channel 240 from, respectively, the first side edge 242 and the second side edge 244. As a result, the width of the channel 240 progressively decreases to a minimum and then progressively increases when moving in the downstream direction past the flow restriction 260. It may be preferred that the width changes (and, thus, the open cross-sectional area changes) are progressive such that fluid flow passing the flow restriction 260 can be maintained without the risk of forming dead zones, for example, bubble or particle trapping, and closed recirculation. It is not preferred that the flow restrictions result in pooling, collecting or other phenomena that may inhibit mixing of the fluids passing through the channel 240. The use of a waveform protrusion as a flow restriction may provide the preferred progressively decreasing and increasing change in open cross-sectional area of the channel. It may further be preferred that the channel height through the length of the flow restriction 260 in the downstream direction be substantially constant, although this is not required.

The depicted flow restriction 260 includes a first element 262 extending into the width of the channel 240 from the edge 242 and a second element 264 extending into the width of the channel 240 from the opposing edge 244. Although two elements 262 and 264 are included, some flow restrictions 260 may include only one such element.

A portion of another exemplary mixer according to the present invention is depicted in the cross-sectional view of FIG. 8. The mixer includes a channel 340 formed between a cover 320 and a base 330. Unlike the mixers described in connection with FIGS. 1-7, at least some of the features of the channel 340 that assist in mixing may be located in, e.g., the cover 320. The base 330 forms the bottom surface 346 of the channel 340 and the cover 320 includes an interior surface 322 that forms the top surface of the channel 340.

The channel 340 also includes a set of flow obstacles 350 that protrude into the channel 340. In the depicted embodiment, the flow obstacles 350 protrude into the channel 340 from the interior surface 322 of the cover 320 rather than the from the bottom surface 346 of the channel 340 as in the exemplary embodiments described above. Like the flow obstacles described elsewhere herein, the flow obstacles 350 are preferably positioned at intermediate locations between the edges of the channel 340. The flow obstacles 350 also preferably obstruct fluid flow through the channel 340, with each flow obstacle forcing a portion of the fluid to change direction within the channel 340.

The channel 340 may also preferably include flow restrictions 360 positioned along the channel 340 between the inlet and the outlet of the channel 340. Each flow restriction 360 of the set of flow restrictions preferably has a downstream length over which the open cross-sectional area of the channel 340 decreases and increases when moving the downstream direction 319.

The variations depicted in FIG. 8 are provided to illustrate the concept that the flow obstacles and the flow restrictions can be provided from any surface defining the open cross-sectional area of the channels in mixers of the present invention. The design and placement of the flow obstacles are selected to obstruct fluid flow through the channel, with each flow obstacle forcing a portion of the fluid to change direction within the channel. The design and placement of the flow restrictions preferably results in progressive decreasing and increasing the open cross-sectional area of the channel as described herein, regardless of their location.

Although the body 10 containing one or more mixers according to the present invention as depicted in FIG. 1 is generally flat and the downstream direction of flow defined by the mixing structure may be described as following a straight linear path. The mixing structures of the invention may alternatively be located within a curved body 410 as depicted in, e.g., FIG. 9. If the body containing the mixing structure is curved, the downstream direction of flow defined by the mixing structure may be described as following a curvilinear path through the body.

The bodies containing static mixers of the present invention may be rigid or flexible (where a flexible body may be manipulated between flat or non-flat (i.e., curved) without significant permanent deformation of the body and without destroying the integrity of the channels in the mixing structure). For example, in some embodiments, a body containing one or more of the static mixers of the present invention may be manipulated into a curved shape during use to assist in processing, reduce the volume needed for the mixer, etc.

Although the static mixers may be used in many different fluid applications, it may be preferred that the static mixers of the present invention be used in fluid systems that incorporate one or more of the static mixers.

FIG. 10 depicts one exemplary integrated fluid system 500 that is integrated into one unitary body 502 and that incorporates at least one static mixer according to the present invention and channels that can be used to fluidly connect the different features in the system 500. The depicted fluid system 500 includes two chambers 570 & 572 that feed into one mixer 510a provided in the fluid system 500. The mixer 510a may preferably, but not necessarily, be a static mixer constructed according to the present invention. Although two chambers 570 & 572 are included in the fluid system 500, other fluid systems 500 may include only one such chamber or more than two chambers that feed into the mixer 510a. In the depicted embodiment, the chambers may be used to introduce one or more samples and one or more reagents into the mixer 510a. In some embodiments, one of the chambers may be dedicated to introducing samples to the mixer 510a while the other chamber may be used to introduce one or more reagents into the mixer (although in some fluid systems, samples may be premixed or loaded with one or more reagents, carrier fluids, etc. into one or both of the chambers).

After passing through the first mixer 510a, the mixed fluid may be collected in an intermediate chamber 574 located downstream of the mixer 510a. The intermediate chamber 574 may, in some embodiments, contain one or more reagents that may be contacted by the mixed fluid entering the intermediate chamber 574. That contact may preferably result in at least some of the one or more reagents in the intermediate chamber 574 being taken up into the mixed fluid.

The fluid system 500 of FIG. 10 also includes a second mixer 510b located downstream of the intermediate chamber 574. The second mixer 510b may, for example, be used to mix one or more reagents taken up in the intermediate chamber 574 with the mixed fluid that was delivered into the intermediate chamber 574 from the first mixer 510a. The second mixer 510b may be of the same design as the first mixer 510a or it may be of a different design. In some fluid systems, both mixers 510a and 510b may be constructed according to the present invention, while in other fluid systems only one of the mixers may be manufactured according to the principles of the present invention.

The fluids that exit the second mixer 510b may be delivered into another chamber 576 located downstream from the second mixer 510b in the fluid system 500. It may be preferred that the chamber 576 contain one or more additional reagents that may be combined with the mixed fluid exiting the second mixer 510b. In some embodiments, for example, the chamber 576 may include one or more reagents that assist in detection of one or more analytes within the mixed fluid delivered into the chamber 576.

The fluid system 500 depicted in FIG. 10 may also preferably include a collection chamber 578 located downstream of the chamber 576. The collection chamber 578 may be used as, e.g., a waste chamber to collect materials from the chamber 576.

Fluid movement through the various features in the fluid system 500 may be supplied using any suitable technique or techniques through one or more channels extending between the different features in the system 500. For example, fluid movement may be driven by gravity, capillary forces, centrifugal forces (if, e.g., the fluid system 500 is rotated), etc. In some instances, the fluid system 500 may include one or more pumps that may function to either drive fluid through the various features using positive pressure or, alternatively, to pull fluids through the structures using negative pressure (e.g., vacuum) developed downstream of the feature or features through which fluid is to be pulled. The pumps may include a power source (e.g., a battery, etc.) or the pumps used in connection with the present invention may be manually powered. Examples of some other potentially suitable manually powered pumps may include, e.g., devices that include resilient cavities that can be compressed and, when returning to their pre-compression states, provide a vacuum force at the inlet of the pump (e.g., bulbs, hemovacs, etc.).

Although not depicted in FIG. 10, the fluid system 500 may also include one or more fluid control features such as valves to control the flow through the various features. For example, it may be preferred that any fluids introduced into the chambers 570 and 572 upstream of the first static mixer 510a be held in the chambers until the fluids are ready to be simultaneously introduced into the mixer 510a. The valves may include physical structures (e.g., sacrificial membranes, ball valves, gate valves, etc.) that are physically opened or they may be fluidic features capable of providing fluid flow control (e.g., capillary valves that prevent fluid flow using, e.g., surface tension, etc.).

### Applications of Static Mixers

In one application, the mixer can be used as a component of a device that can perform an immunoassay, such as a lateral flow immunoassay. One or more mixers can be molded in a substrate that also provides molded features to hold reagents for an assay.

In one embodiment, the device could have a molded chamber upstream of the mixer to hold a binding agent, such as a conjugate antibody, and a molded feature downstream of the mixer, which provides a defined location where a capture agent, such as a capture antibody, can be immobilized.

Alternatively, the device could be designed with molded features that allow inserts upstream and/or downstream of the mixer. The inserts would consist of a substrate functionalized with a binding agent, such as conjugate and/or a capture antibody. Appropriate substrates used as inserts could include filter membranes such as nylon, nitrocellulose, PTFE, PVDF, polysulphone; or films such as polypropylene, polyester, polyethylene, and polycarbonate. Binding agents, such as capture and/or conjugate antibodies, may be immobilized on these membranes or film inserts using coating processes typically used for nitrocellulose-based immunoassays, such as those processes described by BioDot, Inc (Irvine, Calif.).

The device can also include molded features to allow for collection and containment of waste fluid downstream of the capture zone. A molded feature for this purpose could be a reservoir filled with a cellulose wicking material in capillary contact with the microfluidic system capable of holding a volume of fluid between 10 and 1000 uL. The wicking material can be chosen to have specific physical properties (i.e. porosity) that will allow not only containment of the waste fluid, but control of the capillary flow rates in the microfluidic device.

The device described above would be used in a manner similar to a lateral flow immunoassay. A given analyte can be introduced in the inlet port of the device upstream of the chamber containing the binding agent, such as conjugate antibody. The analyte-containing fluid then passes through or over the binding agent (e.g., conjugate antibody), allowing the binding agent to diffuse into the fluid stream. The fluid stream can pass through the static mixer as described herein which will facilitate for conjugation of the binding agent to the target analyte. Once mixed, the fluid containing the binding agent/target analyte complex can pass through or over the capture zone where the binding agent/target analyte complex will be captured by another binding agent, e.g., the immobilized capture antibody, thus forming the final immunoassay sandwich. Finally, the remaining fluid stream can enter and collect in the waste chamber. An optional readout determining the presence or absence of a complete immunoassay-sandwich could be based on visual or instrument-based detection, depending on the choice of labels used for the binding agents.

In a second embodiment, the device described above could incorporate parallel fluidic paths on a single molded substrate to allow for the simultaneous detection of multiple analyte targets or to allow the inclusion of control tests. Each fluidic path could contain one or more of the static mixers described herein. The fluidic paths could feed from a single inlet or multiple inlets, depending on the requirements of the immunoassay of interest.

In another embodiment, the device could also include molded features upstream of the chamber for the binding agent, for example a conjugate antibody, to allow for incorporation of a sample preparation. For example, a chamber holding a lysing agent or other chemical treatments, could be incorporated upstream of the of the binding agent in order to liberate analytes, such as protein targets from a cell, that would otherwise not be accessible to the binding agent.

In some cases, it may be advantageous to include one or more mixer elements between a sample preparation chamber and the binding agent's location to increase the efficiency of the sample treatment (e.g., lysis efficiency). In other cases, the sample preparation may use paramagnetic beads to isolate and concentrate a sample. It may be possible to mold features in the device that will hold these types of beads as well as the magnets necessary to effect the separations when necessary along the flow path. Another possibility may be to include molded features that will incorporate filtration elements based on size exclusion to prepare the sample.

The complete disclosure of the patents, patent documents, and publications cited in the Background, the Detailed Description of Exemplary Embodiments, and elsewhere herein are incorporated by reference in their entirety as if each were individually incorporated.

Exemplary embodiments of this invention are discussed and reference has been made to possible variations within the scope of this invention. These and other variations and modifications in the invention will be apparent to those skilled in the art without departing from the scope of the invention, and it should be understood that this invention is not limited to the exemplary embodiments set forth herein. Accordingly, the invention is to be limited only by the claims provided below and equivalents thereof.

1. A static mixer comprising a mixing structure formed within a body, wherein fluid flowing through the mixing structure defines a flowpath comprising a downstream direction through the mixing structure from an inlet to an outlet, and wherein the mixing structure further comprises:
a channel extending from the inlet to the outlet, the channel comprising a bottom surface and a top surface located opposite the bottom surface, the channel further comprising a first edge and a second edge, wherein the first edge and the second edge extend along a length of the channel on opposite sides of the channel;
a set of flow obstacles protruding into the channel, wherein the flow obstacles are positioned at intermediate locations between the first edge and the second edge of the channel;
wherein the channel comprises a set of flow restrictions positioned along the channel between the inlet and the outlet, wherein each flow restriction of the set of flow restrictions comprises a downstream length over which an open cross-sectional area of the channel decreases and increases when moving in the downstream direction.

2. A static mixer according to claim 1, wherein the channel comprises a set of waveform protrusions positioned along the channel, wherein each waveform protrusion extends across the channel from the first edge to the second edge, and wherein, when moving in the downstream direction, a height of the channel between the bottom surface and the top surface decreases and then increases.

3. A static mixer according to claim 1, wherein the channel comprises a set of waveform protrusions positioned along the channel, wherein, when moving in the downstream direction, a width of the channel between the first edge and the second edge decreases and then increases.

4. A static mixer according to claim 1, wherein the open cross-sectional area of the channel is substantially constant between the waveform protrusions.

5. A static mixer according to claim 1, wherein the bottom surface, the set of flow obstacles, and the set of waveform protrusions are all formed in a completely integral, one-piece base.

6. A static mixer according to claim 5, wherein the top surface of the channel is formed in discrete cover that is attached to the base.

7. A static mixer according to claim 1, wherein, along the downstream direction, the channel comprises a serpentine flow path between the first side surface and the second side surface.

8. A static mixer according to claim 1, wherein a plurality of flow obstacles of the set of flow obstacles comprise a terminal end that does not reach an opposing surface of the channel, such that fluid can pass between the terminal end of the flow obstacle and the opposing surface of the channel.

9. A static mixer according to claim 8, wherein the terminal surface comprises a ramp surface, wherein the ramp surface is inclined relative to the opposing surface.

10. A static mixer according to claim 9, wherein the ramp surface comprises an upstream edge that is lower than a downstream edge.

11. A static mixer according to claim 1, wherein the top surface of the channel is a flat, featureless surface.

12. A static mixer according to claim 1, wherein the first edge of the channel comprises a first side surface located between the bottom surface and the top surface, and wherein the second edge of the channel comprises a second side surface located between the bottom surface and the top surface, wherein the second side surface is located opposite the first side surface.

13. A static mixer according to claim 1, wherein, in a plane oriented orthogonal to the downstream direction of the static mixer, the channel comprises a maximum height between the top surface and the bottom surface of 250 micrometers or less.

14. A static mixer according to claim 1, wherein, in a plane oriented orthogonal to the downstream direction of the static mixer, the channel comprises a maximum height between the top surface and the bottom surface of 100 micrometers or less.

15. A static mixer according to claim 1, wherein, in a plane oriented orthogonal to the downstream direction of the static mixer, the channel comprises a maximum width between the first edge and the second edge of 500 micrometers or less.

16. A static mixer according to claim 1, wherein, in a plane oriented orthogonal to the downstream direction of the static mixer, the channel comprises a maximum width between the first edge and the second edge of 250 micrometers or less.

17. A static mixer according to claim 1, wherein, in a plane oriented orthogonal to the downstream direction of the static mixer, the channel comprises a maximum open cross-sectional area of 50,000 square micrometers or less.

18. A static mixer according to claim 1, wherein, in a plane oriented orthogonal to the downstream direction of the static mixer, the channel comprises a maximum width between the first edge and the second edge and a maximum height between the top surface and the bottom surface, and further wherein a ratio of the maximum width to the maximum height is 1 or more.

19. A static mixer according to claim 1, wherein, in a plane oriented orthogonal to the downstream direction of the static mixer, the channel comprises a maximum width between the first edge and the second edge and a maximum height between the top surface and the bottom surface, and further wherein a ratio of the maximum width to the maximum height is 2 or more.

20. A static mixer according to claim 1, wherein, in a plane oriented orthogonal to the downstream direction of the static mixer, the channel comprises a maximum width between the first edge and the second edge and a maximum height between the top surface and the bottom surface, and further wherein a ratio of the maximum width to the maximum height is 2 or more and 5 or less.

21. A static mixer according to claim 1, wherein the channel comprises a length measured along the downstream direction from the inlet to the outlet of 100 millimeters or less.

22. A static mixer according to claim 1, wherein the body comprises a flexible body.

23. A static mixer according to claim 1, wherein the downstream direction comprises a curvilinear path that varies in three dimensions.

24. An integrated fluid system comprising, in one unitary body, at least the following components:
a static mixer according to claim 1;
a first chamber located upstream of the static mixer;
a second chamber located downstream of the static mixer, and
fluid connection channels extending between the static mixer, the first chamber, and the second chamber.

25. An immunoassay device, comprising the static mixer of claim 1.
